(19) United States
(12) Patent Application Publication
(10) Pub. No.: US 2008/0107780 A1
(43) Pub. Date: May 8, 2008

(54) SUGAR COATINGS AND METHODS THEREFOR

(75) Inventors: John KRESEVIC, New Windsor, NY (US); Sheetal KULKARNI, Haskell, NJ (US); Xiuying LIU, Glen Rock, NJ (US); Nizamuddin BAKSH, Paramus, NJ (US); Robin ENEVER, New City, NY (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/935,114

(22) Filed: Nov. 5, 2007

Related U.S. Application Data
(60) Provisional application No. 60/864,718, filed on Nov. 7, 2006.

(51) Int. Cl. A61K 9/36; A23G 3/50

(52) U.S. Cl. 426/103; 426/293; 426/654

(57) ABSTRACT

The invention provides sugar-containing compositions suitable for use in coating solid preparations such as tablets, pills, granules and grains. Methods of using such coatings are provided, as are solid dosage forms coated with the compositions. In some embodiments, the methods provide sugar coated tablets comprising conjugated estrogens, and a progestin, for example medroxyprogesterone acetate.

This application claims the benefit of priority of U.S. Provisional Appl. Ser. No. 60/864,718, filed Nov. 7, 2006, which is hereby incorporated by reference in its entirety.

### FIELD OF THE INVENTION

The invention is directed generally to the field of pharmaceutical formulations. More specifically, the invention relates to sugar-containing compositions suitable for use in coating solid preparations such as tablets, pills, granules and grains. Methods of using such coatings are provided, as are solid dosage forms coated with the compositions. In some embodiments, the methods provide sugar coated tablets comprising conjugated estrogens, and a progestin, for example medroxyprogesterone acetate.

### BACKGROUND OF THE INVENTION

Sugar coating has been widely known for decades and is still utilized in the confectionary industry to a large extent. Unfortunately, sugar-coating is a multi-step and tedious process, and is highly dependent on the use of skilled manpower. Thus, the pharmaceutical industry under-utilizes this lost process for drug development. However, new processing technologies have remedied several of the aspects of sugar coating that caused reliance on the individual skill of persons involved in the coating process, and it is being utilized as another option for the pharmaceutical industry once again. Thus, compared to modern film coating procedures, sugar coating has kept until today its significance as a coating procedure despite its disadvantages, such as its longer processing times.

Classically, sugar coating was performed in solid, rotating stainless steel pans. However, during the modernization of the process, pharmaceutical sugar coating moved away from solid pans to perforated pans in order to reduce the amount of sucrose crystals adhering to tablets during the long coating cycles. The perforated coating pans have also been shown to be advantageous in allowing for more efficient air passage across the bed of tablets in the pan for more elegant tablets and slightly shorter processing times.

The sugar coating process consists of various steps such as sealing, sub-coating which is optional and is also considered as inert filling to round the edges out prior to final coats and or color coat. In the past, a smoothing step also would have been employed to prepare the surface for color, as well as a finish stage process for a very elegant surface finish over the color stage. The first step, which is the sealing step, involves application of an alcoholic solution of a resin, such as shellac. Sufficient coating is applied to the tablet bed in order to cover all surfaces of the tablet. Talc or Calcium Sulfate is used to prevent sticking of the tablet to the pan. Most of the weight gain increase occurs in the next step, which is the sub-coating step, also known as the inert filling step. If an active overcoat is desired it is possible to introduce an additional step here where an active drug is added in the coating suspension. See U.S. Pat. Nos. 5,547,948, 5,759,576 and 5,759,577 to R. J. Barcomb. This may be either to separate two interacting actives, one in the tablet core and one in the coating or to have an immediate release of the active ingredient from the coat. Water-soluble dyes or water insoluble lakes can be incorporated in the color-coating step in order to make the tablet aesthetically appealing. Finally, wax such as Carnauba Wax dispersed in an organic solvent such as Mineral Spirit is applied over the coated tablets in polishing pans to give a final gloss to the tablets.

One of the drawbacks of traditional sugar coating is the necessity for sub-coating or the inert fill stage in order to provide a uniform and smooth surface for the overcoat. The shape of the tablet plays a very important role—while such might be less important in case of nearly round tablets, it has been necessary for tablets with edges. A round, deep convex tablet is much easier to coat than an oval tablet; however, it really depends on the suspension characteristics. This procedure is often time consuming and tedious.

A further difficulty with sugar coatings is the tendency for cracking of the coating to occur. Potential reasons for cracking include low mechanical strength of coating, exacerbated by inadequate plasticization or binder or excessive pigmentation; differences in thermal or moisture expansion characteristics between the core and the coating; and extended elastic recovery of core after compaction.

It can be seen that there exists a need for improved sugar coatings, and processes for their preparation that address the disadvantages of traditional sugar coatings. The present invention is directed to these, as well as other, ends.

### SUMMARY OF THE INVENTION

The present invention provides compositions and processes for sugar coating of tablets, and the like, that remove the necessity for sub-coating or the inert fill stage in order to provide a uniform and smooth surface for the overcoat, and allow coating directly over tablet cores. Accordingly, the processes of the invention are more economical and more efficient than traditional sugar coating processes. Another advantage of the present processes is a reduction of cracking of coated tablets.

The invention further provides solid dosage forms that contain a coating in accordance with compositions described herein. Thus, according to the present invention there is provided a solid dosage form comprising a core material, and at least one coating disposed thereon, wherein the coating comprises:
from about 30 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, at least one plasticizer in an amount of up to about 5 weight %;
optionally, a glidant, in an amount of up to about 3 weight %; and
optionally, a therapeutic agent in an amount of up to about 10 weight %.

In some further embodiments, the coating includes or consists of:
from about 70 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, from about 0.5 weight % to about 1.5 weight % of at least one plasticizer;
optionally, a glidant, in an amount of up to about 1 weight %; and
optionally, a therapeutic agent in an amount of up to about 5 weight %.

In some embodiments, the ratio of the weight percent of binder to the weight percent of diluent/binder in the coating is from about 8:1 to about 12:1; or is about 10:1.

In some embodiments, the ratio of the weight percent of binder to the weight percent of surfactant in the coating is from about 12:1 to about 20:1; or is from about 15:1 to about 18:1; or is from about 16:1 to about 17:1.

In some embodiments, the ratio of the weight percent of diluent/binder to the weight percent of surfactant in the coating is from about 1.2:1 to about 2:1; or is from about 1.5:1 to about 1.8:1.

In some embodiments, the ratio of the weight percent of binder; to the weight percent of surfactant; to the weight percent of diluent/binder in the coating is about 10:0.6:1.

In some further embodiments, the coating includes or consists of:
from about 87 weight % to about 94 weight % of at least one sugar;
from about 0.4 weight % to about 0.6 weight % of at least one diluent/binder;
from about 0.28 weight % to about 0.32 weight % of at least one surfactant;
from about 4 weight % to about 6 weight % of at least one binder;
from about 0.5 weight % to about 1.5 weight % of at least one plasticizer;
optionally, a glidant, in an amount of up to about 1 weight %; and
optionally, a therapeutic agent in an amount of up to about 5 weight %.

In some such embodiments, the ratio of the weight percent of binder to the weight percent of diluent/binder in the coating is from about 8:1 to about 12:1; or is about 10:1.

In some further such embodiments, the ratio of the weight percent of binder to the weight percent of surfactant in the coating is from about 12.5:1 to about 20:1; or is from about 15:1 to about 18:1; or is from about 16:1 to about 17:1.

In some further such embodiments, the ratio of the weight percent of diluent/binder to the weight percent of surfactant in the coating is from about 1.25:1 to about 2:1; or is from about 1.5:1 to about 1.8:1.

In some further such embodiments, the ratio of the weight percent of binder; to the weight percent of surfactant; to the weight percent of diluent/binder in the coating is about 10:0.6:1.

In some embodiments, plasticizer, the glidant, and the therapeutic agent are each present in the coating.

In some embodiments, the core material includes conjugated estrogens.

In some embodiments, the dosage forms further include one or more additional coatings, for example a color coating and/or a polish coating.

In some embodiments, the present invention provides aqueous compositions useful for preparing dosage forms of the invention. In some embodiments, the aqueous compositions include a solids component and water. In some embodiments, the solids component includes or consists of:

at least one sugar, in an amount of from about 30 weight % to about 95 weight % of the solids component;

at least one diluent/binder, in an amount of from about 0.3 weight % to about 0.8 weight % of the solids component;

at least one surfactant, in an amount of from about 0.28 weight % to about 0.4 weight % of the solids component;

at least one binder, in an amount of from about 4 weight % to about 6 weight % of the solids component;

optionally, at least one plasticizer, in an amount of up to about 5 weight % the solids component;

optionally, at least one glidant, in an amount of up to about 3 weight % the solids component; and

optionally, a therapeutic agent, in an amount of up to about 10 weight % the solids component; wherein the water is present in an amount of from about 30% to about 50% by weight of the aqueous composition.

In some further embodiments, the solids component includes or consists of:
from about 70 weight % to about 95 weight % of the sugar;
from about 0.3 weight % to about 0.8 weight % of the diluent/binder;
from about 0.28 weight % to about 0.4 weight % of the surfactant;
from about 4 weight % to about 6 weight % of the binder;
optionally, from about 0.5 weight % to about 1.5 weight % of the plasticizer;
optionally, up to about 1 weight % of the glidant; and
optionally, up to about 5 weight % of the therapeutic agent.

In some further embodiments, the solids component includes or consists of:
from about 87 weight % to about 94 weight % of the sugar;
from about 0.4 weight % to about 0.6 weight % of the diluent/binder;
from about 0.28 weight % to about 0.32 weight % of the surfactant;
from about 4 weight % to about 6 weight % of the binder;
from about 0.5 weight % to about 1.5 weight % of the plasticizer;
optionally, up to about 1 weight % of the glidant; and
optionally, up to about 5 weight % of the therapeutic agent.

In some embodiments of the dosage forms and solids components of the aqueous compositions of the invention, and the sugar includes or consists of sucrose; the diluent/binder includes or consists of microcrystalline cellulose; the surfactant includes or consists of sodium lauryl sulfate; and the binder includes or consists of a polyvinylpyrrolidone. In some such embodiments, the plasticizer, when present, includes or consists of a polyethylene glycol; the glidant, when present, includes or consists of silica; and the therapeutic agent, when present, includes or consists of a progestin, for example medroxyprogesterone acetate.

In some embodiments, in the solids components of the aqueous compositions of the invention, the ratios of the weight percents of the binder, surfactant and binder are as described above.

The present invention further provides processes for preparing a solid dosage form. In some embodiments, the processes include coating a core material with an aqueous composition of the invention. In some embodiments, the processes further include applying one or more additional coats to the coated core material, such as a color coat, a polish coat, or both a color coat and a polish coat.

In some embodiments of each or the dosage forms of the invention, the core material includes at least one therapeutic agent. In some embodiments, the estrogen is conjugated estrogens. In some embodiments, the coating of the solid dosage forms includes a progestin, for example medroxyprogesterone acetate.

The invention also provides products of the processes described herein.

### DESCRIPTION OF THE DRAWINGS

FIGS. 1-5 show a baffle design and coating pan incorporating the baffles amenable to the preparation of dosage forms according to the invention.

### DETAILED DESCRIPTION OF THE INVENTION

Concentrations, amounts, percentages, and other numerical data may be expressed or presented herein in a range format. It is to be understood that such a range format is used merely for convenience and brevity and thus, should be interpreted flexibly to include not only the numerical values explicitly recited as the limits of the range, but also, to include each of the individual numerical values or sub-ranges encompassed within that range as if each numerical value and sub-range is explicitly recited.

As an illustration, a concentration range of “about 1 weight % to about 10 weight %” should be interpreted to include not only the explicitly recited concentration of about 1 weight % to about 10 weight %, but also individual concentrations and the sub-ranges within the indicated range. Thus, included in this numerical range are individual concentrations such as 2 weight %, 5 weight %, and 8 weight %, and sub-ranges such as from 1 weight % to 3 weight %, from 5 weight % to 9 weight %, etc. The same principle applies to ranges reciting only one numerical value.

Similarly, an open ended range recited as “less than about 10 weight %” or “up to about 5 weight %” should be interpreted to include all of the values and ranges as elaborated above. Furthermore, it is understood that functional limitations may exist for limits not expressly recited by an open ended range, and that such limitations are included inherently as part of the disclosure of the present application, though not expressly recited. Such an interpretation should apply regardless of the breadth of the range or the characteristics being described.

In some embodiments, the present invention provides solid dosage forms that include a core material, and at least one coating disposed thereon. In some embodiments, the coating includes or consists of:
from about 30 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, at least one plasticizer in an amount of up to about 5 weight %;
optionally, a glidant, in an amount of up to about 3 weight %; and
optionally, a therapeutic agent in an amount of up to about 10 weight %.

In some further embodiments, the coating includes or consists of:
from about 70 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, from about 0.5 weight % to about 1.5 weight % of at least one plasticizer;
optionally, a glidant, in an amount of up to about 1 weight %; and
optionally, a therapeutic agent in an amount of up to about 5 weight %.

In some preferred embodiments, the ratio of the weight percent of binder to the weight percent of diluent/binder in the coating is from about 8:1 to about 12:1, preferably about 10:1.

In some further preferred embodiments, the ratio of the weight percent of binder to the weight percent of surfactant in the coating is from about 12:1 to about 20:1, preferably from about 15:1 to about 18:1; more preferably from about 16:1 to about 17:1.

In some embodiments, the ratio of the weight percent of diluent/binder to the weight percent of surfactant in the coating is from about 1.2:1 to about 2:1; preferably from about 1.5:1 to about 1.8:1.

In some preferred embodiments, the ratio of the weight percent of binder; to the weight percent of surfactant; to the weight percent of diluent/binder in the coating is about 10:0.6:1.

In some further embodiments, the coating includes or consists of:
from about 87 weight % to about 94 weight % of at least one sugar;
from about 0.4 weight % to about 0.6 weight % of at least one diluent/binder;
from about 0.28 weight % to about 0.32 weight % of at least one surfactant;
from about 4 weight % to about 6 weight % of at least one binder;
from about 0.5 weight % to about 1.5 weight % of at least one plasticizer;
optionally, a glidant, in an amount of up to about 1 weight %; and
optionally, a therapeutic agent in an amount of up to about 5 weight %.

In some such embodiments, the ratio of the weight percent of binder to the weight percent of diluent/binder in the coating is preferably from about 8:1 to about 12:1; preferably about 10:1.

In some further such embodiments, the ratio of the weight percent of binder to the weight percent of surfactant in the coating is preferably from about 12.5:1 to about 20:1; preferably from about 15:1 to about 18:1; preferably from about 16:1 to about 17:1.

In some further such embodiments, the ratio of the weight percent of diluent/binder to the weight percent of surfactant in the coating is preferably from about 1.25:1 to about 2:1; preferably from about 1.5:1 to about 1.8:1.

In some preferred embodiments of each of the foregoing dosage forms, the ratio of the weight percent of binder; to the weight percent of surfactant; to the weight percent of diluent/binder in the coating is about 10:0.6:1.

In some embodiments, the invention provides solid dosage forms comprising a core material and at least one coating disposed thereon, wherein the coating includes or consists of:
from about 87 weight % to about 94 weight % of at least one sugar;
from about 0.4 weight % to about 0.6 weight % of microcrystalline cellulose;
from about 0.28 weight % to about 0.32 weight % of sodium lauryl sulfate;
from about 4 weight % to about 6 weight % of a polyvinylpyrrolidone;
from about 0.5 weight % to about 1.5 weight % of at least one plasticizer;
optionally, a glidant, in an amount of up to about 1 weight %; and
medroxyprogesterone acetate in an amount of up to about 5 weight %;
   and wherein the core material comprises conjugated estrogens. In some such embodiments, sugar includes or consists of sucrose. In further such embodiments, the sugar includes or consists of sucrose; the plasticizer includes or consists of polyethelene glycol; and the binder includes or consists of Povidone K25. In some further such embodiments, the ratio of the weight percent of binder; to the weight percent of surfactant; to the weight percent of diluent/binder is about 10:0.6:1.

The compositions of the present invention are particularly suitable for use in coating a core material to produce a solid dosage form. The term “core material” refers to any tablet, caplet, particle, micronized particle, particulate, pellet, pill, core, powder, granule, granulate, small mass, seed, specks, spheres, crystals, beads, agglomerates, mixtures thereof and the like. Typically, the preferred core material will be in a form sufficiently stable physically and chemically to be effectively coated in a system that involves some movement, as for example, a tablet in a perforated coating pan.

In a preferred embodiment, the core material is present in the form of a tablet. As used herein, the term “tablet” refers to a solid pharmaceutical dosage form containing a therapeutic agent with or without suitable diluents and prepared by either compression or molding methods, such as are well known to those of ordinary skill in the art. Suitable methods of forming tablets are described, for example, in Edward M Rudnick, et al., “Oral Solid Dosage Forms,” in Remington: The Science and Practice of Pharmacy, 20^{th} Ed., Chap. 45, Alfonso R. Gennaro, ed., Philadelphia College of Pharmacy and Science, Philadelphia, Pa. (2000), herein incorporated by reference in its entirety. In some preferred embodiments, the core material is a tablet formed by compression methods.

Most frequently, the core material will comprise at least one therapeutic agent, and at least one pharmaceutically acceptable excipient. As used herein, the term “therapeutic agent” refers to a substance which is capable of exerting a therapeutic biological effect in vivo. The therapeutic agents may be neutral or positively or negatively charged. Examples of suitable pharmaceutical agents include, inter alia, diagnostic agents, pharmaceuticals, drugs, synthetic organic molecules, proteins, peptides, vitamins, and steroids. The term “pharmaceutically acceptable,” as used herein, refers to materials that are generally not toxic or injurious to a patient when used in the compositions of the present invention, including when the compositions are administered by the oral route. The term “patient,” as used herein, refers to animals, including mammals, preferably humans. “Excipients,” as that term is used herein, refers to ingredients that provide bulk, impart satisfactory processing and compression characteristics, help control the dissolution rate, and/or otherwise give additional desirable physical characteristics to the core material. Included within this term, for example, are diluents, binders, lubricants and disintegrants well known to those of ordinary skill in the art, as described, for example, in the Handbook of Pharmaceutical Excipients, American Pharmaceutical Association, Washington, D.C. and The Pharmaceutical Society of Great Britain, London, England (1986), herein incorporated by reference in its entirety. Suitable excipients may include, for example, cellulosic material, such as, hypromellose, hydroxypropylcellulose (HPC), hydroxyethylcellulose (HEC), carboxymethylcellulose, microcrystalline cellulose, ethyl cellulose, methyl cellulose, and their derivatives and salts; other organic compounds, such as polyethylene glycol (PEG), talc, lactose and other sugars (as described above), acacia, dextrin, alginic acid, ethylcellulose resin, gelatin, guar gum, methylcellulose, pregelatinized starch, sodium alginate, starch, zein, polyvinylpyrrolidone, vinylpyrrolidine-vinyl acetate copolymer, vinyl acetate-crotonic acid copolymer and ethyl acrylate-methacrylate acid copolymer; plasticizers, such as propylene glycol, glycerin, trimethylolpropane, polyethylene glycol polymers, dibutyl sebacate, acetylated monoglycerides, diethylphthalate, triacetin, glyceryltriacetate, acetyltriethyl citrate and triethyl citrate; and lubricants, such as talc, magnesium stearate, calcium stearate, stearic acid, hydrogenated vegetable oils, magnesium lauryl sulfate, sodium benzoate, a mixture of sodium benzoate and sodium acetate, sodium chloride, leucine, and Carbowax® 4000.

A wide variety of therapeutic agents may be utilized in the core material. Specific examples of therapeutic agents include, but are not limited to: acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenytoin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenytoin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, vitamin K-S (II) and zafirlukast. Additional therapeutic agents include abacavir, acebutolol, acrivastine, alatrofloxacin, albuterol, albendazole, alfentanil, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, apomorphine, astemizole, atenolol, atropine, azathioprine, azelastine, azithromycin, baclofen, benethamine, benidipine, benzhexol, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclo-cycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyl-imidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, modafinil, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxatidine, roxithromycin, salbutamol, saquinavir, selegiline, sertraline, sibutramine, sildenafil, sparfloxacin, spiramycins, stavudine, sufentanil, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₅, vitamin K₆, vitamin K₇, zafirlukast, zolmitriptan, zolpidem and zopiclone. Of course, any of the foregoing therapeutic agents may be included in the coating composition, as discussed previously, and any of the therapeutic agents discussed with regard to the coating composition alternatively may be included in the core material.

The core material may be designed for delivering therapeutic agents intended to be delivered over a sustained period of time. The following are representative of such therapeutic agents: anti-inflammatory, antipyretic, anti-spasmodics or analgesics such as indomethacin, diclofenac, diclofenac sodium, codeine, ibuprofen, phenylbutazone, oxyphenbutazone, mepirizole, aspirin, ethenzamide, acetaminophen, aminopyrine, phenacetin, butylscopolamine bromide, morphine, etomidoline, pentazocine, fenoprofen calcium, naproxen, selecxip, valdecxip, and tolamadol, anti-rheumatism drugs such as etodolac, anti-tuberculoses drugs such as isoniazide and ethambutol hydrochloride, cardiovascular drugs such as isosorbide dinitrate, nitroglycerin, nifedipine, barnidipine hydrochloride, nicardipine hydrochloride, dipyridamole, amrinone, indenolol hydrochloride, hydralazine hydrochloride, methyldopa, furosemide, spironolactone, guanethidine nitrate, reserpine, amosulalol hydrochloride, lisinopril, metoprolol, pilocarpine, and talcetin, antipsychotic drugs such as chlorpromazine hydrochloride, amitriptyline hydrochloride, nemonapride, haloperidol, moperone hydrochloride, perphenazine, diazepam, lorazepam, chlorodiazepoxide, adinazolam, alprazolam, methylphenidate, myrnasipran, peroxetin, risperidone, and sodium valproate, anti-emetics such as metoclopramide, lamocetron hydrochloride, granisetron hydrochloride, ondansetron hydrochloride, and azacetron hydrochloride, antihistamines such as chlorpheniramine maleate and diphenhydramine hydrochloride, vitamins such as thiamine nitrate, tocopherol acetate, cycothiamine, pyridoxal phosphate, cobarnamide, ascortic acid, and nicotinamide, anti-gout drugs such as allopurinol, colchicine, and probenecide, anti-Parkinson's disease drugs such as levodopa and selegrine, sedatives and hypnotics such as amobarbital, bromuralyl urea, midazolam, and chloral hydrate, antineoplastics such as fluorouracil, carmofur, acralvidine hydrochloride, cyclophosphamide, and thiodepa, anti-allergy drugs such as pseudoephedrine and terfenadine, decongestants such as phenylpropanolamine and ephedorine, diabetes mellitus drugs such as acetohexamide, insulin, tolbutamide, desmopressin, and glipizide, diuretics such as hydrochlorothiazide, polythiazide, and triamterene, bronchodilators such as aminophylline, formoterol fumarate, and theophylline, antitussives such as codeine phosphate, noscapine, dimorfan phosphate, and dextromethorphan, anti-arrhythmics such as quinidine nitrate, digitoxin, propafenone hydrochloride, and procainamide, topical anesthetics such as ethyl aminobenzoate, lidocaine, and dibucaine hydrochloride, anti-convulsants such as phenytoin, ethosuximide, and primidone, synthetic glucocorticoids such as hydrocortisone, prednisolone, triamcinolone, and betamethasone, antiulceratives such as famotidine, ranitidine hydrochloride, cimetidine, sucralfate, sulpiride, teprenone, plaunotol, 5-aminosalicylic acid, sulfasalazine, omeprazole, and lansoprazol, central nervous system drugs such as indeloxazine, idebenone, thiapride hydrochloride, bifemelane hydrocide, and calcium homopantothenate, antihyperlipoproteinemics such as pravastatin sodium, simvastatin, lovastatin, and atorvastatin, antibiotics such as ampicillin hydrochloride, phthalylsulfacetamide, cefotetan, and josamycin, BPH therapeutic agents such as tamsulosin hydrochloride, doxazosin mesylate, and terazosin hydrochloride, drugs affecting uterine motility such as branylcast, zafylcast, albuterol, ambroxol, budesonide, and reproterol, peripheral circulation improvers of prostaglandin I derivatives such as beraprost sodium, anticoagulants, hypotensives, agents for treatment of cardiac insufficiency, agents used to treat the various complications of diabetes, peptic ulcer therapeutic agents, skin ulcer therapeutic agents, agents used to treat hyperlipemia, tocolytics, etc. The therapeutic agent can be used in its free form or as a pharmaceutically acceptable salt. Moreover, one or a combination of two or more therapeutic agents may be present in the core material.

In some embodiments, the therapeutic agent in the core material includes conjugated estrogens. “Conjugated estrogens” (CE) as used herein includes both natural and synthetic conjugated estrogens, such as the compounds described in the United States Pharmacopeia (USP 23), as well as other estrogens so considered by those skilled in the art. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-alpha and beta-dihydroequilin, equilenin, 17-alpha and beta-dihydroequilenin, estrone, 17-beta-estradiol, and their sodium sulfate esters.

Although CE are typically a mixture of estrogenic components, such as estrone and equilin, the core material may be formulated to either utilize such a mixture, or to include only selected or individual estrogenic components. These CE may be of synthetic or natural origin. Examples of synthetically produced estrogens include, inter alia, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, estropipate and ethinyl estradiol. The alkali metal salts of 8,9-dehydroestrone and the alkali metal salts of 8,9-dehydroestrone sulfate ester, as described in U.S. Pat. No. 5,210,081, which is herein incorporated by reference, also may be used. Naturally occurring CE are usually obtained from pregnant mare urine and then are processed and may be stabilized. Examples of such processes are set forth in U.S. Pat. Nos. 2,565,115 and 2,720,483, each of which is incorporated herein by reference.

Many CE products are commercially available. Preferred among these is the naturally occurring CE product known as Premarin® (Wyeth, Madison, N.J.). Another commercially available CE product prepared from synthetic estrogens is Cenestin® (Duramed Pharmaceuticals, Inc., Cincinnati, Ohio). The specific CE dose included in the core material may be any dosage required to achieve a specific therapeutic effect, and may vary depending on the specific treatment indicated, and on the specific CE included in the tablet. However, in general, dosages of CE included in the tablet can range from about 0.1 mg CE/dosage form to about 5.0 mg CE/dosage form, with dosages of from about 0.3 mg CE/dosage form to about 2 mg CE/dosage form preferred. In some embodiments, the dosage of CE is from about 0.3 mg CE/dosage form, about 0.45 mg CE/dosage form, about 0.625 mg CE/dosage form, about 0.9 mg CE/dosage form, or about 1.25 mg CE/dosage form. Viewed alternatively, based on the total weight of the solid dosage form, on a dry weight basis, the amount of CE/dosage form may range from about 0.05 weight % to about 1.0 weight %, with amounts of from 0.1 weight % to about 0.3 weight % preferred.

In some embodiments, the invention is directed to compositions suitable for use in coating a core material, as described above. The compositions include a solvent, preferably water, and a solids component, that can optionally include a therapeutic agent. Preferably, the composition is in the form of an aqueous suspension, obtained by combining from about 30 weight % to about 98 weight % water, and from about 2 weight % to about 70 weight % of the solids component. In certain embodiments, the composition includes from about 30 weight % to about 50 weight % water and from about 50 weight % to about 70 weight % of the solids component. In one such embodiment, the composition includes about 40 weight % water, and about 60% solids component.

The aqueous compositions of the invention are useful, inter alia, to prepare dosage forms of the invention. The dosage forms can include a core material as described above, and a coat thereon provided by coating the core with an aqueous composition of the invention. Thus, in the description herein, the composition of the solids component of the aqueous compositions of the invention reflects the composition of the coating of the core materials in the dosage forms of the invention.

The solids component contains, inter alia, one or more sugars. As used herein, the term “sugar” refers to any type of simple carbohydrate, such as a mono or disaccharide, either naturally obtained, refined from a natural source, or artificially produced, and includes, without limitation, sucrose, maltose, glucose, fructose, galactose, mannose, mannitol, lactose, trehalose, lactulose, levulose, raffinose, ribose, and xylose. The term “sugar,” as used herein, also includes various “sugar substitutes” widely known to those of ordinary skill in the art of preparing solid dosage forms, such as the polyhydric alcohols (sometimes referred to as “sugar alcohols” or hydrogenated saccharides), for example sorbitol, mannitol, xylitol, and erythritol, and the sugar derivatives of polyhydric alcohols, such as maltitol, lactitol, isomalt, and polyalditol. Accordingly, the recitation of the term “sugar” generically should be interpreted to include such specific compounds, as well as others not expressly recited. In certain embodiments, the solids component of compositions of the invention include at least one sugar that is a mono-or disaccharide, for example, sucrose, dextrose, maltose, glucose, fructose, galactose, mannose, lactose. In some such embodiments, the sugar is sucrose.

Generally, the solids component contains from about 30 weight % to about 95 weight % sugar. In still other embodiments, the solids component contains from about 70 to about 95 weight % sugar. In still other embodiments, the solids component contains from about 87 to about 94 weight % sugar. In still other embodiments, the solids component contains about 91 weight % sugar.

The solids component also contains a diluent/binder. As used herein, the term “dliuent/binder” is intended to mean a compound that is known to function as a diluent, as a binder, or as both a diluent and a binder in pharmaceutical formulations. One preferred diluent/binder is microcrystalline cellulose. Other suitable diluent/binders include other cellulose derivatives, such as hydroxyethylcellulose (HEC), hydroxyethylmethylcellulose (HEMC), powdered cellulose, carboxylmethyl cellulose, hydroxypropylcelluloise (HPC), hydroxymethylpropytlcellulose (HPMC), and other agents that are known to function as diluents and binders in pharmaceutical dosage forms, such as alginic acid, polyethylene oxide, sodium alginate, maltodextrin, pregelatinized starch zein, acacia gum, guar gum, carbomer, and starch. In some embodiments, the diluent/binder can in be a sugar, provided that it is not the same sugar employed as the sugar component of the coatings of the invention. Generally, the diluent/binder is present in the solids component in an amount of from about 0.3 weight % to about 0.8 weight %. In some embodiments, the diluent/binder is present in an amount of from about 0.4 weight % to about 0.6 weight %. In some preferred embodiments, the diluent/binder is present in an amount of about 0.5 weight % of the solids component.

It will be appreciated that the diluent/binder is employed in the present coatings in relatively small amounts compared to the typical use of a diluent in pharmaceutical formulations. It has been discovered in accordance with the present invention that the presence of the diluent/binder in such amounts, in conjunction with the amounts of surfactant and binder described herein, provide significant benefits to the coatings of the invention in terms of, for example, appearance, reduced cracking and dissolution characteristics.

The solids component also contains a surfactant. The surfactant can be selected from those known to be useful in the art, including for example metal alkyl sulfates such as sodium lauryl sulfate, polyoxyethylene sorbitan fatty acid esters, polyoxyethylene castor oil derivatives, docusate sodium, sugar esters of fatty acids and glycerides of fatty acids, alkali salts of fatty acids, polyethylene-propylene glycol copolymers, phosphatidylcholine, polyethylene glycol fatty acid esters, polyoxyethylene alkyl ethers, sorbitan fatty acid esters, polyethylene glycol fatty acid esters, docusate sodium, sorbitan esters and emulsifying wax. In some preferred embodiments, the surfactant includes or consists of sodium lauryl sulfate.

Generally, the surfactant is present in the solids component in an amount of from about 0.28 weight % to about 0.4 weight %. In some embodiments, the surfactant is present in an amount of from about 0.28 weight % to about 0.32 weight %. In some preferred embodiments, the surfactant is present in an amount of about 0.3 weight % of the solids component.

The solids component further contains at least one binder. The binder can be any of the variety of agents that are known to function as binders in pharmaceutical dosage forms, provided that the diluent/binder and the binder are not the same. Suitable binders include gelatin, polyvinylpyrrolidone (PVP), hydroxypropylmethylcellulose (HPMC), pregelatinized starch, plain starch, hydroxypropylcellulose (HPC) and carboxymethylcellulose (CMC). In some preferred embodiments, the binder includes or consists of a polyvinylpyrrolidone, for example Povidone K25.

Generally, the binder is present in the solids component in an amount of from about 4 weight % to about 6 weight %. In some preferred embodiments, the binder is present in an amount of about 5 weight % of the solids component.

The solids component of the compositions also can optionally contain one or more plasticizers. Suitable plasticizers also are well known to those skilled in the art, and include, for example, propylene glycol, glycerin, trimethylolpropane, polyethylene glycol (PEG) polymers, dibutyl sebacate, acetylated monoglycerides, diethylphthalate, triacetin, glyceryltriacetate, acetyltriethyl citrate and triethyl citrate. In certain embodiments, a PEG polymer is used. Such polymers are available commercially by grades of average molecular weight such as PEG 100 to PEG 4,000. In some preferred embodiments, the plasticizer includes or consists of PEG 400. Other suitable plasticizers include dibutyl phthalate, tributyl citrate, dimethyl phthalate, pyrrolidones, DBS (dibutyl sebacate), DBP (dibutyl phthalate), DEP (diethyl phthalate), DMP (dimethyl phthalate), PVAP (polyvinyl acetate phthalate), TEC (triethyl citrate), TBC (tributyl citrate), glycerin, glycerin monostearate, sorbitol, mineral oil, triethanolamine, triacetin, stearic acid, acetyltributyl citrate, acetyltriethyl citrate, and dextrin. When present, the plasticizer is generally employed in the solids component in an amount of up to about 5 weight %. In some embodiments, the plasticizer is present in an amount from about 0.5 weight % to about 1.5 weight %. In some preferred embodiments, the plasticizer is present in an amount of about 1 weight % of the solids component.

The solids component of the compositions also can optionally contain one or more glidants. Suitable glidants also are well known to those skilled in the art, and include, for example, silica, dibasic calcium phosphate, magnesium carbonate, magnesium oxide, calcium silicate, silicon dioxide, silicon dioxide aerogels, hydrous magnesium calcium silicate (Talc), magnesium trisilicate, and magnesium silicate. In some preferred embodiments, the glidant includes or consists of silica, for example Cab-O-Sil®. When present, the glidant is generally employed in the solids component in an amount of up to about 3 weight %. In some embodiments, the glidant is present in an amount of up to about 1 weight %. In some preferred embodiments, the glidant is present in an amount of about 0.5 weight % of the solids component.

The solids component of the compositions also can optionally contain one or more therapeutic agents, which can be any of those as defined previously. In one nonlimiting example, the solids component can include one or more hormonal steroids, such as medroxyprogesterone acetate, levonorgestrel, gestodene, medrogestone, estradiol, estriol, ethinylestradiol, mestranol, estrone, dienestrol, hexestrol, diethylstilbestrol, progesterone, desogestrel, norgestimate, hydroxyprogesterone, norethindrone, norethindone acetate, norgestrel, megestrol acetate, methyltestosterone, ethylestrenol, methandienone, oxandrolone, trimegestone, dionogest, and the like. Additionally, tissue selective progesterones and/or progesterone antagonists, which may or may not have the typical steroidal functionality, may be present in the composition. These include, but are not limited to: RU-486 (mifepristone), ZK 98 299 (onapristone), ZK-137316 (Schering AG, Berlin), ZK-230211 (Schering AG, Berlin), and HRP-2000 (17-acetoxy-[11β-(4-N,N-dimethylaminophenyl)]-19-norpregna-4,9-diene-3,20-dione). Where desired, estrogenic steroids and progestogenic steroids may be used in combination.

In addition to the coating compositions described previously, the present invention also is directed to solid dosage forms comprising a core material and one or more coatings disposed thereon, as each element has been described heretofore. In certain embodiments, the dosage form is a coated tablet. In some embodiments, the solid dosage forms comprise from about 30 weight % to about 70 weight % of the core material, and from about 30 weight % to about 70 weight % of the coating, and in still further embodiments, from about 40 weight % to about 60 weight % of the core material and from about 40 weight % to about 60 weight % of the coating. The solid dosage forms also can optionally include one or more additional coats, for example, a further sugar coating as described herein, disposed either on top of the coating previously described, or in between the core and coating. The solid dosage form also can include one or more a color coats and/or polish coats. In some embodiments, the color coat constitutes from about 0.5 weight % to about 15 weight % of the dosage form, and/or a polish coat that constitutes from about 0.01 weight % to about 5 weight % of the dosage form.

Any of the aforementioned therapeutic agents can be utilized in the core or the sugar coating. In some preferred embodiments, the invention provides coated tablets that utilize conjugated estrogens, such as the conjugated estrogens desiccation with lactose, in the core material, and a progestin, for example medroxyprogesterone acetate, in the sugar coating. In certain embodiments, the conjugated estrogens are present in an amount from about 0.1 mg CE/dosage form to about 5.0 mg CE/dosage form, or from about 0.3 mg CE/dosage form to about 2 mg CE/dosage form. In some embodiments, the dosage of CE is from about 0.3 mg CE/dosage form, about 0.45 mg CE/dosage form, about 0.625 mg CE/dosage form, about 0.9 mg CE/dosage form, about 1.25 mg CE/dosage form, or 2.5 mg CE/dosage form. Preferably, the CE is present in the tablet core, which can be coated with a coating composition as described herein, in an amount of from about 30 weight % to about 75 weight %, based on the total weight of the solid dosage form, to produce a coated tablet. An optional color coat and/or polish coat also may be applied, as described previously.

It has been found in accordance with the present invention that control of the ratio of the amount of binder, for example polyvinylpyrrolidone, and diluent/binder, for example microcrystalline cellulose, affords significant advantages in terms of properties of the dosage forms, including processing, appearance and dissolution characteristics thereof. Additional advantages, including further improvements to the aforementioned properties, are afforded by controlling the amount of surfactant employed in the coating compositions. Accordingly, in some preferred embodiments, the ratio of the weight percent of binder to the weight percent of diluent/binder in the coating is from about 8:1 to about 12:1; or is about 10:1. In some further embodiments, the ratio of the weight percent of binder to the weight percent of surfactant in the coating is from about 12:1 to about 20:1; or is from about 15:1 to about 18:1; or is from about 16:1 to about 17:1. In some further embodiments, the ratio of the weight percent of diluent/binder to the weight percent of surfactant in the coating is from about 1.2:1 to about 2:1; or is from about 1.5:1 to about 1.8:1. In some further embodiments, the ratio of the weight percent of binder; to the weight percent of surfactant; to the weight percent of diluent/binder in the coating is about 10:0.6:1.

In some embodiments, in the coating of the solid dosage form, as described in any of the preceding embodiments, or combinations thereof:
the sugar comprises sucrose;
the diluent/binder comprises microcrystalline cellulose;
the surfactant comprises sodium lauryl sulfate; and
the binder comprises a polyvinylpyrrolidone.

In some embodiments, coating of the solid dosage form, as described in any of the preceding embodiments, or combinations thereof:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

In some embodiments, the therapeutic agent of the solid dosage form comprises a progestin. In some embodiments, the therapeutic agent of the solid dosage form comprises medroxyprogesterone acetate.

In some embodiments, the plasticizer, the glidant, and the therapeutic agent are each present in the coating.

In some embodiments, the solid dosage form further comprises a color coating.

In some embodiments, in the solids component of the aqueous composition, as described in any of the preceding embodiments, or combinations thereof:
the sugar comprises sucrose;
the diluent/binder comprises microcrystalline cellulose;
the surfactant comprises sodium lauryl sulfate; and
the binder comprises a polyvinylpyrrolidone.

In some embodiments, in the solids component of the aqueous composition, as described in any of the preceding embodiments, or combinations thereof:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

In some embodiments, the therapeutic agent of the aqueous composition comprises a progestin. In some embodiments, the therapeutic agent of the aqueous composition comprises medroxyprogesterone acetate.

In some embodiments, the plasticizer, the glidant, and the therapeutic agent are each present in the solids component.

One advantage of the present invention is that a wide variety of tablet cores, prepared according to the various processes known in the art, can readily be coated with the coating compositions of the present invention to provide a coated tablet core. Thus, some embodiments, the present invention is directed to processes that include providing a tablet core and applying, e.g., by spraying, onto the core a sugar coating composition, as previously described.

In certain embodiments, the sugar coating composition is disposed directly onto the tablet core without a need for intervening sealing layers, as are used typically in traditional sugar-coating methods. If desired, however, a sealing layer, such as shellac and other agents known to those in the art, may be applied to the tablet core prior to application of the sugar coating composition. In some embodiments, the sugar coating composition contains a therapeutic agent, such as medroxyprogesterone acetate, as previously described, and is disposed directly onto the tablet core without a non-therapeutic agent containing sugar coat being first applied, or an intervening sealing layer.

While the processes of the present invention further may include the steps of spraying a color coat and/or polish coat onto the sugar coat, such steps are optional, and all of the coating steps may be carried out in a single coating pan or sequentially. Also, the step of printing a logo, trademark, word, symbol or the like optionally may be included in the processes of the present invention. Printing may be performed by any of the methods well known to those skilled in the art.

Thus, the process of the present invention may comprise the steps of providing tablet cores, placing the tablet cores into a coating pan, such as a perforated coating pan commonly utilized in sugar coating applications such as a perforated pan with side-vents, then spraying the tablet cores with a sugar coating composition as described herein. In some embodiments, the spraying is performed using incremental shots of the coating composition, until a desired weight of coating is applied. A similar procedure can be employed to provide any desired color coats or polish coats. Spray techniques for coating tablets are well known to those of skill in the art, and are described, for example, in Stuart C. Porter, “Coating of Pharmaceutical Dosage Forms,” Remington: The Science and Practice of Pharmacy, 20^{th} Ed., Chap. 46, Alfonso R. Gennaro, ed., Philadelphia College of Pharmacy and Science, Philadelphia, Pa. (2000), herein incorporated by reference in its entirety.

The invention also is directed to the products of such processes, including for example, a coated tablet core, or such a coated core having one or more additional color and/or polish coats, as described above.

The processes of the present invention are much simpler, less labor intensive, and less reliant upon operator expertise than the traditional sugar-coating techniques known in the prior art. Additionally, due to the unique combination of ingredients utilized in the sugar coating composition, the coated tablets produced by the processes of the present invention are remarkably hard, durable, and resistant to cracking, even when highly hygroscopic tablet cores are utilized. In some embodiments, a plurality of tablet cores coated with the sugar coating compositions of the present invention contain cracking in less than 6 percent of the coated tablet cores. In further such embodiments, the percentage of cracks is about 1 to about 5 percent; and in still further embodiments, less than 1 percent. Additionally, the coating provides an excellent barrier to prevent the release of odors from the tablet core, and to prevent atmospheric elements from reaching and degrading the therapeutic agent(s) in the tablet core. Thus, the coating compositions and processes described herein are particularly well suited for preparing solid dosage forms that utilize therapeutic agents or other materials having strong odors, such as, sulfur-containing compounds, in the core material.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of Premarin/MPA (0.45/1.5 mg) Coated Tablets

Coated tablets were prepared by coating a tablet core material containing conjugated estrogens (Premarin®; 0.45 mg) with an aqueous coating suspension (60% solids; 40% water) containing medroxyprogesterone acetate (MPA) to provide 1.5 mg of MPA per coated tablet, according to the procedures below. The coated tablet was then coated with a color coating suspension to provide a color coating, and then further coated with a polishing suspension to provide a polish coat. The compositions of the coating suspension; the color coating suspension, and the polish suspension are shown in Table 1 below:

TABLE 1
Composition of Coating Suspensions

| | | |
|---|---|---|
| | Amt/tablet | |
| Description | (mg) | % of Solids |

Aqueous Coating Suspension

| | | |
|---|---|---|
| Medroxyprogesterone Acetate, USP | 1.59 | 1.50 |
| Micronized @ 100%^{a} | | |
| Sucrose, NF | 96.672 | 91.2 |
| Microcrystalline Cellulose, NF | 0.53 | 0.50 |
| Sodium Lauryl Sulfate, NF | 0.318 | 0.30 |
| Polyethylene Glycol 400, NF | 1.06 | 1.0 |
| Povidone K25, USP | 5.3 | 5.0 |
| Cab-O-Sil, NF | 0.53 | 0.50 |
| Water, USP, Purified^{b} | 70.67 | — |
| Totals | 106.0 | 100 |

Color Coating Suspension

| | | |
|---|---|---|
| Sucrose, NF | 23.4904 | 94.0 |
| Titanium Dioxide, USP | 0.7692 | 3.1 |
| Povidone K25, USP | 0.6154 | 2.4 |
| Cab-O-Sil, NF | 0.1250 | 0.5 |
| Water, USP, Purified^{b} | 13.4615 | — |
| Totals | 25.0 | 100 |

Polish Coating Suspension

| | | |
|---|---|---|
| Carnauba Wax, NF #120 | 0.222 | 100 |
| Mineral Spirits, Odorless^{b} | 0.469 | — |
| Totals | 0.222 | 100 |

^{a}Indicates that the potency of Medroxyprogesterone Acetate, USP Micronized 100% may vary and the amount in the formulation must be adjusted accordingly. If the amount of the MPA is adjusted, the amount of Sucrose was adjusted accordingly. A 6% overage is included to compensate for the manufacturing loss during coating.
^{b}Removed during processing

### Coating Equipment

Three different types of pans were used: Colton 12″ pan, Compu-Lab19″ pan and Compu-Lab 24″ pan. The pan speed was set at 15-20 rpm for the 12″ pan, 10-12 rpm for the 19″, 12-20 rpm for the 24″ pan. The 12″ Colton pan did not contain any baffles whereas the 19″ and 24″ pans had baffles, which allowed for efficient mixing of the tablets.

In a further procedure, the process was scaled up in a GCX-1000 pan (manufactured by Glatt Air Technologies—inside diameter of 40″), which is used for coating production-scale batches.

The filled tablets were then colored using the color suspension containing titanium dioxide, as shown above.

### Preparation of MPA Coating Suspension

The MPA coating suspension was prepared using following steps:

1. Purified water was added to an appropriately sized jacketed container. While mixing with a high shear mixer, the water was heated to 65° C.±5° C. and the Sucrose was added. The mixture was reheated to 65° C. and mixed until all the sucrose was dissolved.

2. The solution was cooled to 40-45° C. Using a high shear mixer, the Polyethylene Glycol, Povidone K25, Microcrystalline Cellulose, and Cab-O-Sil were slowly added to the vortex. The excipients were mixed until suspension was complete. The suspension was mixed using a high shear mixer for another minute one or more times as necessary to ensure complete mixing.

3. While mixing with the high shear mixer, the above suspension was cooled to 35-39° C. and the sodium lauryl sulfate and MPA were slowly added. The suspension was mixed continuously using a low shear mixer, while maintaining the tank temperature at 35° C. to 39° C. during the entire application process.

### Application of MPA Coating Suspension Using Comp-U-Lab or Colton Coaters

1. To the pre-selected size (12, 19, and 24 inches pans were utilized) perforated (solid pan in case of Colton design) coating pan, an appropriate quantity of hydrogel Premarin tablet cores were loaded.

2. The inlet temperature was set at 40° C. and inlet airflow at 75 cfm. The tablets were preheated to about 30° C., dew point 11° C. and exhaust temperature at 35° C. Colton conditions are manually set.

3. With the pan rotating at pre selected rpm's (pending pan size), incremental shots of MPA coating suspension were applied by a syringe (and/or measuring devise) until an average tablet weight gain of 106 mg was achieved. Each shot was followed by a tumbling jog cycle of 180-300 seconds (no air through the coating pan) followed by a drying phase of 60-180 seconds.

### Color Coating Suspension Preparation

1. Purified Water was added to an appropriately sized jacketed container. While mixing with a high shear mixer, the water was heated to 65° C.±5° C. and the Sucrose added. The suspension was reheated to 65° C., and stirring continued until all the sucrose was dissolved.

2. The Povidone and Titanium Dioxide were added, and the suspension mixed using a high shear mixer to insure homogeneous suspension.

3. The Cab-O-Sil was added, and the suspension mixed using a high shear mixer to make a homogeneous suspension.

4. The suspension was cooled to 35-39° C. Mixing was performed continuously using a low shear mixer, while maintaining the tank temperature at 35° C. to 39° C. during the entire application process.

### Color Coating Suspension Application

1. An appropriate quantity of Premarin/MPA filled tablets were loaded to a 24″ perforated coating pan installed in a Comp-U-Lab coater.

2. The inlet temperature was set at 40° C. and inlet airflow at 75 cfm. The tablets were preheated to about 30° C., dew point 11° C. and exhaust temperature at 35° C.

3. With the pan rotating at about 18 rpm, incremental shots of color coating suspension were applied, until an average tablet weight gain of 25 mg was achieved. Each shot was followed by a tumbling jog cycle of 180-300 seconds (no air through the coating pan) followed by a drying phase of 60-180 seconds.

### Preparation and Application of Polish Coating

1. The polish coating suspension was prepared by suspending the Carnauba Wax, NF, #120 in the Mineral Spirits, Odorless with vigorous stirring.

2. The polish suspension was applied to the rolling tablets, with rolling continued until a satisfactory gloss was obtained.

### Dissolution Studies

The dissolution was determined in an apparatus having 900 mL of 0.54% Sodium Lauryl Sulfate (SLS) in water, equipped with a paddle rotating at 75 rpm. A filtered sample of the dissolution medium was taken at specified times. The release of the active was determined by reversed phase high performance liquid chromatography.

### Weight Variation

Samples of approximately 150 tablets were taken at predetermined weight gains during the filling process. Weight variation of 1000 tablets was evaluated using Mocon Automatic Balance Analysis tester (Modern Controls, Inc., Minneapolis, Minn.).

### Integrity and Solvent Permeability (Cracking)

Integrity Test

100 tablets were allowed to slide down a plexiglass tube (one inch I.D.×36 inches at 37 deg.±2 deg.) into a 1 L stainless steel beaker (held at the same angle). This step was repeated four additional times. The same tablets were used for the solvent permeability test.

Solvent Permeability Test

The 100 tablets were placed in a suitable container and sufficient amount of dye solution (D&C Green #6 dissolved in ethyl acetate) was added to cover the tablets. The container was then sealed and allowed to stand for 18-24 hours. After the specified period, the tablets were removed from the dye solution and rinsed several times with clear ethyl acetate to remove any excess dye. The tablets were allowed to dry at ambient (room) temperature. The numbers of tablets showing cracks were then determined by observation.

Appearance

The appearance of 100 tablets was determined visually or under a set of magnifying glasses to observe for surface abnormalities.

### Results

A number of experiments were conducted to examine the effect of varying the microcrystalline cellulose (MCC) level on the dissolution profile of the active. Table 2 shows the concentration of MCC in the sugar coating for each batch. The amount of silicon dioxide in the sugar coating for Batches 1 to 4 was 0%, 0.5%, 0%, and 1%, respectively. The concentration of Povidone (PVP), polyethylene glycol (PEG) and sodium lauryl sulfate (SLS) in the sugar coating for each batch was maintained at 5%, 1% and 0%, respectively. The amount of sucrose in the sugar coating was adjusted from the amount in Table 1 to maintain the desired solids level.

As can be seen in Table 2, as the amount of microcrystalline cellulose was increased from 0.8% MCC (microcrystalline cellulose) to 5.0% MCC the dissolution profile of the active decreased or slowed down. The amount of Povidone was kept constant at 5% in all of these batches.

TABLE 2
Influence of Various Levels of Microcrystalline Cellulose
on the Dissolution of Active in the Tablets

| | Batch | | | |
|---|---|---|---|---|
| | 1 | 2 | 3 | 4 |
| Time (hr) | (0.8% MCC) | (1.2% MCC) | (3.0% MCC) | (5.0% MCC) |
| 0 | 0 | 0 | 0 | 0 |
| 0.25 | 8.99 | 7.07 | 3.59 | 3.1 |
| 0.5 | 22.54 | 19.64 | 10.71 | 10.1 |
| 0.75 | 38.61 | 51.74 | 36.37 | 31.3 |
| 1 | 56.97 | 69.94 | 50.81 | 44.8 |
| Infinity (30 | 111.81 | 110.21 | 99.03 | 100.7 |
| mins at 200 | | | | |
| rpm) | | | | |

A second series of experiments was conducted to examine the effect of varying the concentration of PVP and MCC on the cracking and appearance of the coated formulations. Tables 3 and 4 show the relative concentrations of PVP and MCC in the sugar coating. The amount of PEG in the sugar coating was 0.1% for Batch 15 and 1% for the remaining batches. The amount of silicon dioxide in the sugar coating was 0% for Batch 5 and 0.5% for the remaining batches. The amount of MPA was 1.5% for Batches 5-9, 0.75% for Batch 10, 1.1% for Batches 11-12, and 1.3% for Batches 13-15. No SLS was added to any of the batches. The amount of sucrose was adjusted accordingly to maintain the desired solids level.

As can be seen in Table 3, as the amount of PVP is increased from 5% to 7%, all batches yielded 0% cracking results. The batch containing 5% PVP gave excellent looking tablets as compared to the batch containing 7% PVP in which the tablets appeared to have pinholes.

TABLE 3
Influence of Various Levels of Povidone on the Appearance
and Cracking of the Tablets

| | Batch # | | | | | | | | |
|---|---|---|---|---|---|---|---|---|---|
| | 5 | 6 | 7 | 8 | 9 | 10 | 11 | 12 | 13 |
| % Solids | 64.80% | 65% | 65% | 65% | 65% | 65% | 65% | 60% | 60% |
| % | 8% | 5% | 5% | 8% | 8% | 8% | 8% | 6% | 7% |
| Povidone | | | | | | | | | |
| % MCC | 0.8% | 3% | 7% | 6% | 6% | 6% | 6% | 4% | 3% |
| Pan | 12″ | 12″ | 12″ | 12″ | 12″ | 19″ | 19″ | 19″ | 19 |
| Used | | | | | | | | | |
| % | 10 | 2 | 7 | 1 | 5 | 0 | 0 | 0 | 0 |
| Cracked | | | | | | | | | |

As seen in Table 4, the proportion of PVP and microcrystalline cellulose is significant for reducing cracking and obtaining the appropriate release characteristics of the dosage form.

TABLE 4
Influence of Various Levels of Povidone and
Microcrystalline Cellulose on the Cracking of Tablets

| | Batch | | |
|---|---|---|---|
| | 14 | 15 | 16 |
| | W/ | W/ | W/ |
| | 5% Povidone | 6% Povidone | 7% Povidone |
| % Solids | 60% | 60% | 60% |
| % Microcrystalline | 2% | 1.30% | 3.00% |
| Cellulose | | | |
| Pan Used | 24″ | 24″ | 19″ |
| Appearance of tablets | Excellent | Rough | Pinholes |
| Cracking % | 0 | 0 | 0 |

Although batches 8, 10, 11, and 12 gave low cracking results the high percentage of PVP gave problems in processing. The formulation containing 5% PVP (Kollidon K25) and 0.5% microcrystalline cellulose, which gave good results in terms of cracking, dissolution, and appearance, is preferred.

Although batch 14 in Table 4 gave good results (excellent appearance and no cracking) it did not match the dissolution characteristics of the referenced commercial product as shown in Table 5.

TABLE 5
Influence of various levels of Sodium Lauryl Sulfate
on the Dissolution of MPA in the Sugar Coat of the Tablets

| | 14 | 17 | | | |
|---|---|---|---|---|---|
| | w/0% | w/0.15% | | 19 | |
| | SLS | SLS | 18 | w/0.3% SLS | |
| Time | and | and | w/0.25% SLS | and | |
| (hr) | 2% MCC | 1% MCC | and 0.5% MCC | 0.5% MCC | Reference |
| 0 | 0.00 | 0.00 | 0.00 | 0.00 | 0.00 |
| 0.25 | 4 | 7 | 9 | 11 | 15 |
| 0.5 | 9 | 12 | 14 | 19 | 21 |
| 0.75 | 13 | 15 | 18 | 25 | 27 |
| 1 | 16 | 18 | 22 | 29 | 32 |
| 2 | 26 | 27 | 37 | 40 | 49 |
| 6 | 49 | 47 | 64 | 77 | 67 |
| 12 | 83 | 84 | 95 | 94 | 93 |

An additional series of experiments were conducted to examine the effect of SLS and MCC in the sugar coating. The concentration of PVP, PEG and silicon dioxide in the sugar coating for each batch were maintained at 5%, 1% and 0.5%, respectively. The concentration of MPA in the sugar coating for Batches 14, 17, 18, and 19 were 1.3%, 1.5%, 1.5%, and 1.1%, respectively. The amount of sucrose was adjusted in order to maintain the desired solids level. To achieve the desired appearance and dissolution characteristics, the MCC was decreased from 2% in batch 14 to 0.5% in batches 18 and 19 in order to increase the dissolution profile. PVP remained constant (5%) for batches 14, 17, 18 and 19. To further achieve the desired dissolution characteristics, sodium lauryl sulfate (SLS) was introduced to the formulation to aid in the release of MPA. Increasing the initial concentration of sodium lauryl sulfate from 0.25% in batch 18 to 0.3% in batch 19 provided the desired increase in dissolution of the active. The dissolution profile was compared to that of a commercial batch (reference) containing medroxyprogesterone acetate and it was found that the batch containing 0.3% SLS gave a satisfactory result.

A formulation containing 5% PVP, 0.5% microcrystalline cellulose, 1% polyethylene glycol, 0.5% Cab-O-Sil and 0.3% SLS with 60% solid content was prepared, and gave a similar dissolution profile as the commercial product. This formulation was stable under the conditions (25° C./60% RH and 40° C./75% RH) up to 6 months.

### Scale-Up of Formulation in Glatt GCX-1000

To evaluate if scaling-up would have any effect on the product, the dosage form was prepared using a Glatt GCX-1000 coater (40″ pan size). The procedure was the same as that above for the smaller pans, except for the application of the coating suspension, which was applied according to the following procedure:

### Application of MPA Coating Suspension When Scale-up to GCX-1000

1. Approximately 166,666 hydrogel Premarin® tablet cores were loaded to the GCX-1000 coater pan.

2. The inlet temperature was set at 35° C. and inlet airflow at 250 cfm. The tablets were preheated to about 30° C., dew point 12° C. and exhaust temperature at 30° C.

3. Two Graco guns (Graco; Part number 948-864) were mounted at equal distance on the boom. Hydraulic nozzles (Spraying Systems 11001-SS tips) were installed onto the Graco guns, and a Graco pump (piston pump) was connected to the suspension supply line. The suspension spray pressure was adjusted at a pressure of 80-100 psi in order to produce a fan shape spray that covered the entire tablet bed. With the pan rotating at 10 rpm, incremental shots of MPA coating suspension were sprayed until an average tablet weight gain of 106 mg was achieved. Each shot was followed by a tumbling jog cycle of 180-300 seconds (no air through the coating pan) followed by a drying phase of 60-180 seconds.

Two batches were prepared to compare the reproducibility and robustness of the process. In this study GCX-1000 pan consisted of 4 baffles and two sugar coating guns equipped with hydraulic nozzles, described in more detail below. The dissolution profiles and weight variation were compared to determine the reproducibility of the process. The dissolution testing was done on the final polished tablets. The weight variation testing was done during the filling process at predetermined weight gains. It was found that the batches gave results in terms of dissolution which were comparable to a reference product.

To determine the weight variation during the coating process, three batches were prepared, and the weight variation was tested at 10%, 20%, 30%, 40%, 50%, 60%, 70%, 80%, 90% and 100% weight gains. All three batches yielded a final weight variation of less than 3.5%.

The foregoing data show that the process is robust and reproducible.

### Baffle/Coating Pan Design Used in the Present Studies

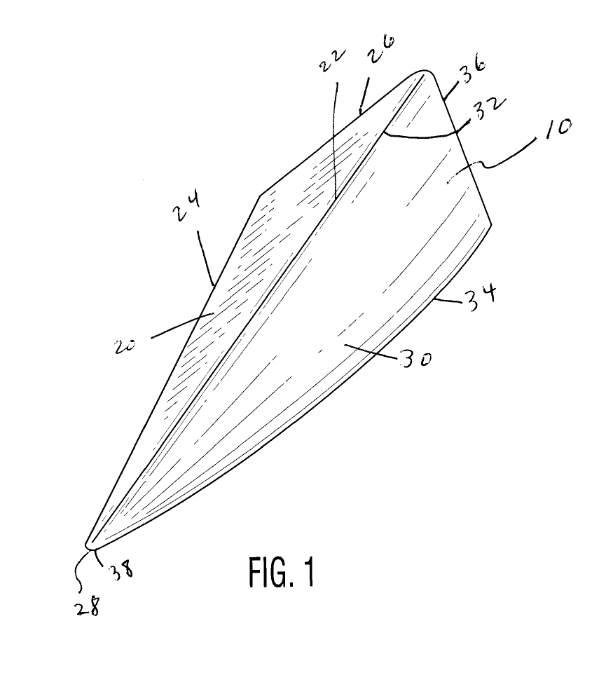

While any coating pan and baffle design can be used in accordance with the present invention, FIGS. 1-5 show the coating pan and baffle design use in the Comp-U-Lab Coater (Example 2) and GCX-1000 scale up studies described herein. See also U.S. Provisional Application Ser. No. 60/864,726, filed Nov. 7, 2006, entitled “Sugar Coating Process and Baffles Therefor”, which is hereby incorporated by reference.

Referring to FIG. 1, baffles (10) were used in the equipment described above. The baffle (10) comprises a first side (20) and a second side (30). The first side (20) comprises three edges: a top edge (22), a bottom edge (24), and a lateral edge (26). The top edge (22) and bottom edge (24) of the first side (20) converge to form a first side tip (28) distal to the lateral edge (26) of the first side (20). In some embodiments, the first side (20) is flat or planar and has no curvature.

Also referring to FIG. 1, the second side (30) also comprises three edges: a top edge (32), a bottom edge (34), and a lateral edge (36). The top edge (32) and bottom edge (34) of the second side (30) converge to form a second side tip (38) distal to the lateral edge (36) of the second side (30). In some embodiments, the second side (30) is curved in a convex manner from the lateral edge (36) to the second side tip (38).

Still referring to FIG. 1, the first side (20) and second side (30) are joined at each of the respective top edges (22) and (32), thus forming a single baffle unit (10) with the first side tip (28) converging with the second side tip (38). In some embodiments, the joining of the sides (20) and (30) can be accomplished by one or more fasteners (not shown) commonly used in the art. For example, the fasteners can be mechanical fasteners such as bolts, screws, hinges, rivets, and the like. In addition, the fasteners can include chemical agents such as glues, epoxys, and the like. Alternately, the joint formed by the first and second sides (20) and (30) can be seamless. Thus, in some embodiments, the first and second sides (20) and (30) can be manufactured as a single integral unit.

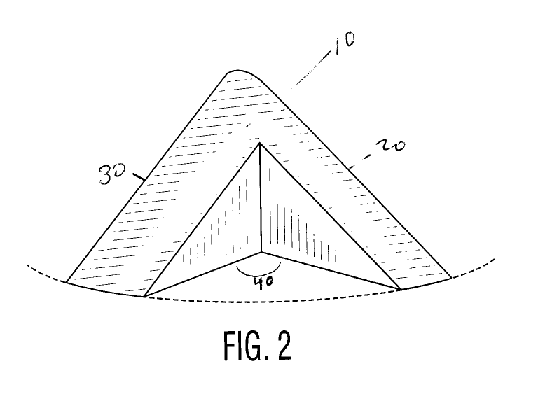

Referring to FIG. 2, the joining of the first and second sides (20) and (30) creates an internal angle (40) no less than about 45°, no less than about 50°, no less than about 55°, no less than about 60°, no less than about 65°, no less than about 70°, no less than about 75°, no less than about 80°, or no less than about 85°. The internal angle (40) formed by the first and second sides (20) and (30) is also no greater than about 120°, no greater than about 115°, no greater than about 110°, no greater than about 105°no greater than about 100°, or no greater than about 95°. In some embodiments, the internal angle is about 90°. In this context, the term “about” means±1°. Referring to FIG. 1, in some embodiments, the length of the baffle from first side tip (28) and/or second side tip (38) along top edges (22) and/or (32) is at least about 6 inches, at least about 8 inches, at least about 10 inches, at least about 12 inches, at least about 14 inches, at least about 16 inches, at least about 18 inches, at least about 20 inches, at least about 24 inches, or longer. In some embodiments, the length of the baffle from first side tip (28) and/or second side tip (38) along top edges (22) and/or (32) is selected such that the gap between the convergence of the first side tip (28) and the second side tip (38) and the end of the cylindrical surface (52) is as described below.

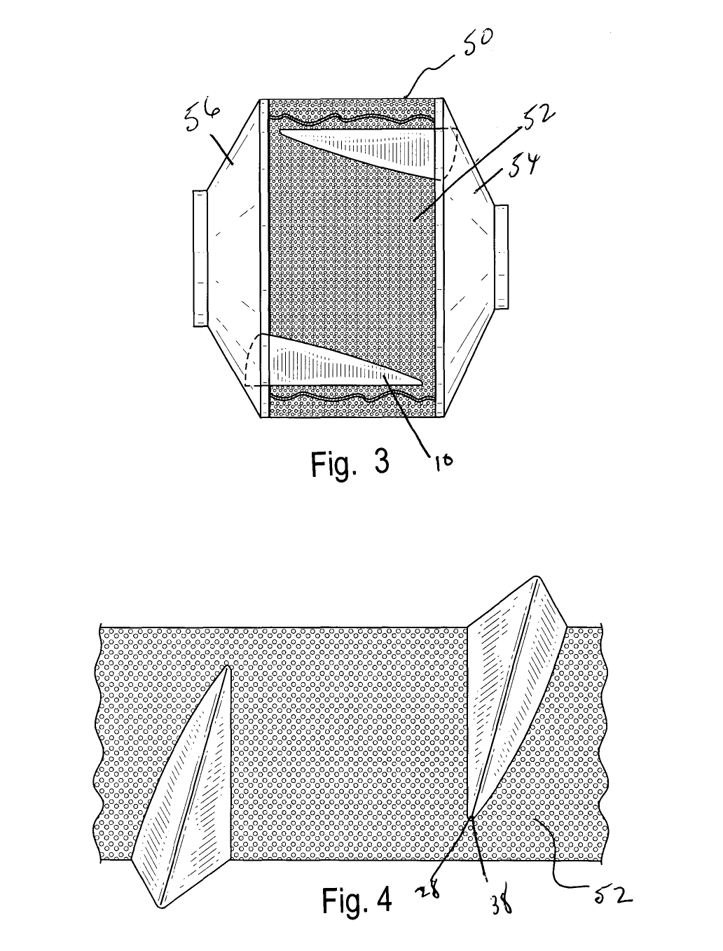

Also referring to FIG. 2, the height of the baffle (10) ranges from about 1 inch to about 8 inches, or from about 5 inches to about 8 inches, or from about 7 inches to about 8 inches, or from about 2 inches to about 4 inches. As used herein, the height of the baffle is the distance between the intersection of the lateral edge (26) and the lateral edge (36), and a surface point. As used herein, a surface point refers to a point on the surface which is midway between the point at which the lateral edge (26) intersects the surface and the point at which the lateral edge (36) intersects the surface, when the baffle is placed on the surface (for example, see FIG. 4). Referring to FIG. 3, in some embodiments, the height of the baffle (10) is about 3 inches. In some embodiments, the height of the baffle (10) is about 6.5 inches. In addition, baffle (10) comprises a length that is no less than about 1/16 inch, no less than about ½ inch, or no less than about 1 inch and no greater than about 4 inches, no greater than about 3 inches, or no greater than about 2 inches shorter than the width of the cylindrical surface (52) of the coating pan (50), thus leaving a gap between the single tip of the baffle (10) and the edge of the cylindrical surface (52) of the coating pan (50). In this context, the term “about” means±¼ inch.

The sides (20) and (30) of baffle (10) can be perforated or non-perforated, and can be made of any material suitable for coating pharmaceutical formulations including, but not limited to, metals such as stainless steel, plastic, fiberglass, Teflon™, and the like. In some embodiments, the surfaces of the sides (20) and (30) are smooth.

In some embodiments, the invention provides a coating pan (50). Referring to FIG. 3, the coating pan (50) comprises a cylindrical surface (52) for receiving a pharmaceutical formulation, an outer wall (54) in contact with one end of the cylindrical surface (52), an inner wall (56) in contact with the other end of the cylindrical surface (52), and at least one baffle (10) as described above. Referring to FIG. 4, the lateral edges (26) and (36) of the sides (20) and (30) of the baffle (10) contact the inner wall (56) or outer wall (54) of the coating pan (50). The bottom edges (24) and (34) of the sides (20) and (30) forming baffle (10) contact the cylindrical surface (52) of the coating pan (50). The coating pan (50) can comprise at least one, at least two, at least three, at least four, at least five, or at least six baffles (10). The baffles (10) can be fastened to the coating pan (50) by any means known to the skilled artisan including, for example, those means described above.

In some embodiments, the tip of at least one baffle (10) formed by the convergence of the first side tip (28) and the second side tip (38) does not extend the entire width of the cylindrical surface (52). Referring to FIG. 4, this leaves a gap between the convergence of the first side tip (28) and the second side tip (38) and the end of the cylindrical surface (52). In some embodiments, the tip of all baffles (10) formed by the convergence of the first side tip (28) and the second side tip (38) does not extend the entire width of the cylindrical surface (52).

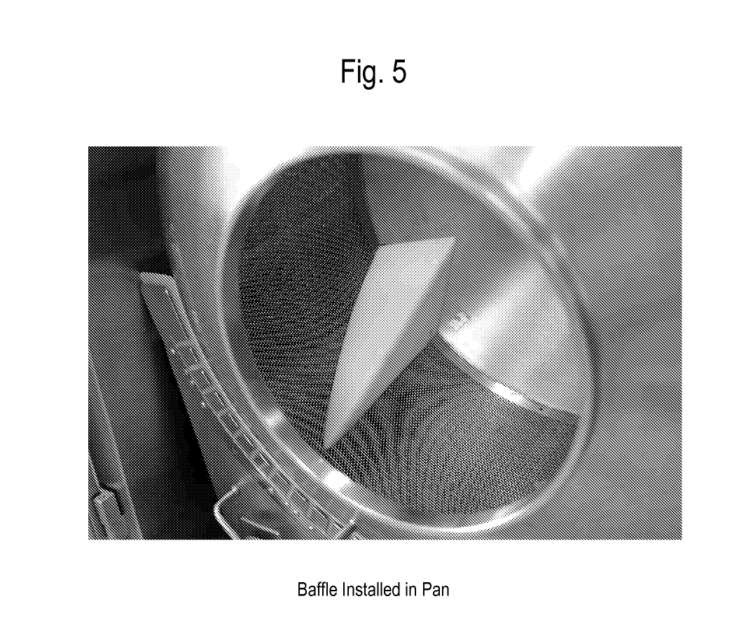

In some embodiments, the gap between the convergence of the first side tip (28) and the second side tip (38) and the end of the cylindrical surface (52) is a distance that is from about 2% to about 50% of the length of the baffle from first side tip (28) and/or second side tip (38) along top edges (22) and/or (32); or from about 2% to about 30% of such length; or from about 2% to about 20% of such length; or from about 2% to about 15% of such length; or from about 10% to about 15% of such length, or from about 12% to about 13% of such length, or from about 2% to about 10% of such length. In some embodiments, the gap is about 12.5% of such length.

Where two or more baffles (10) are present within a coating pan (50), in some embodiments, at least two of the baffles (10) are oriented in the opposite direction. Referring to FIG. 3, the two baffles (10) are oriented such that the lateral edges (26) and (36) of one baffle (10) is contacting the inner wall (56) of the coating pan (50) while the lateral edges (26) and (36) of the other baffle (10) is contacting the outer wall (54) of the coating pan (50). This orientation is also depicted in FIGS. 4 and 5.

In some embodiments, as depicted in FIG. 4, the flat side (20) of the baffle (10) is aligned perpendicularly with the cylindrical surface (52) of the coating pan (50). In other embodiments, the flat side (20) of the baffle (10) can be aligned at any desired angle with the cylindrical surface (52) of the coating pan (50).

In some embodiments, the invention provides a coating apparatus (not shown) comprising a coating pan (50) described above. Coating apparati are well known to the skilled artisan and are commercially available. Suitable coating apparati include, but are not limited to, a 24″ Comp-U-Lab coater (Thomas Engineering, Inc., Hoffman Estates, Ill.) and GCX-1000 coater.

The baffles can be prepared by standard methods for manufacturing a baffle. For example, a template of the baffle (10), such as a cardboard, wood or plastic template, can be created using the contour of the coating pan (50). A baffle material, such as any of the materials described above, can be cut and shaped according to the template. In some embodiments, the shaping of the baffle (10) can be accomplished using a lathe. The baffles (10) can be fastened by any means to the coating pan (50). In some embodiments, the baffles (10) are screwed to the coating pan (50) through pre-existing perforated bed holes.

### Example 2

### Coating Compositions

### Exemplary Coating of Pharmaceutical Formulations Using Baffles Described in FIGS. 1-5

Oval biconvex shaped hydrogel-based Premarin tablets with 0.412 inch×0.225 inch×0.034 inch dimension were used for the coating trials. The tablets contain 0.375% of Conjugated Estrogens, 15% Microcrystalline cellulose (Avicel PH 101), 48.51% Lactose Monohydrate Spray Dried, 27.5% HPMC K100M CR, and 0.25% Magnesium Stearate and had an average weight of 120 mg with a related standard deviation in the range of 0.5 to 1.4%. The hardness of tablet cores ranged from 7 to 10 scu.

Several characteristics of the coated pharmaceutical formulation were observed and monitored including, for example, the physical appearance, percentage of cracked sugar coats, weight variation (at different weight gains), and content uniformity of MPA of resulting tablets.

TABLE 6
Composition of Filler Coating Suspension

| | Input/Tablet |
|---|---|
| Description | (mg) |
| Premarin 0.45 mg tablet core | 120.0 |
| Medroxyprogesterone Acetate, | 1.59 |
| USP, Micronized @100% (A) | |
| Sucrose, NF | 96.672 |
| Microcrystalline Cellulose, NF | 0.53 |
| Sodium Lauryl Sulfate, NF | 0.318 |
| Polyethylene Glycol 400, NF | 1.06 |
| Povidone K25, USP | 5.3 |
| Cab-O-Sil, NF | 0.53 |
| Water, USP, Purified (B) | 70.67 |

(A) Indicates that the potency of the Medroxyprogesterone Acetate, USP, Micronized 100% may vary and the amount in the formulation must be adjusted accordingly. If the amount of MPA is adjusted, the amount of Sucrose will be adjusted accordingly. A 6% overage is included to compensate for the manufacturing loss during coating.
(B) Indicates removed during processing.

TABLE 7
Composition of Color Coating Suspension

| Description | Input/Tablet (mg) |
|---|---|
| Premarin/MPA | 226 |
| (0.45/1.5 mg) Filled | |
| Tablet | |
| Sucrose, NF | 23.4904 |
| Titanium Dioxide, | 0.7692 |
| USP | |
| Povidone K25, USP | 0.6154 |
| Cab-O-Sil, NF | 0.1250 |
| Water, USP, | 13.4615 |
| Purified (A) | |

(A) Indicates removed during processing.

TABLE 8
Composition of Polishing Suspension

| Description | Input/Tablet (mg) |
|---|---|
| Premarin/MPA (0.45/1.5 mg) | 251 |
| colored Tablet | |
| Carnauba Wax, NF, | 0.222 |
| #120 | |
| Mineral Spirits, | 0.469 |
| Odorless (A) | |

(A) Indicates removed during processing.

### Manufacturing Process

### MPA Filler Suspensions Preparation

The MPA filler suspensions were prepared using following steps:

1) Add water, purified in an appropriately sized jacketed container; while mixing with a high shear mixer, heat the water to 65° C.±5° C. and add the sucrose; reheat to 65° C.; mix until all the sucrose is dissolved.

2) Cool the above solution to 40-45° C.; slowly add to the vortex using a high shear mixer, the Polyethylene Glycol, Povidone K25, Microcrystalline Cellulose, and Cab-O-Sil; mix above solution for another 1 minute using a high shear mixer.

3) While mixing with the high shear mixer, cool the above suspension to 35-39° C. and slowly add Sodium Lauryl Sulfate and MPA.

4) Mix continuously using a low shear mixer, while maintaining the tank temperature at 35° C. to 39° C. during the entire application process.

### MPA Filler Suspension Application When Using Comp-U-Lab Coater

1) To the 24″ perforated coating pan with the different designed baffles, load approximately 33,333 hydrogel Premarin tablet cores.

2) Set inlet temperature at 40° C. and inlet airflow at 75 cfm; preheat the tablets to about 30° C., dew point 11° C. and exhaust temperature at 35° C.

3) With the pan rotating at 18 rpm, apply incremental shots of MPA filler suspension by a syringe until an average tablet weight gain of 106 mg is achieved; each shot is followed by a tumbling jog cycle of 180-300 seconds (no air through the coating pan) followed by a drying phase of 60-180 seconds.

### MPA Filler Suspension Application When Scale-up to GCX-1000

1) To the GCX-1000 coater pan with the different designed baffles, load approximately 166,666 hydrogel Premarin tablet cores.

2) Set inlet temperature at 35° C. and inlet airflow at 250 cfm; preheat the tablets to about 30° C., dew point 12° C. and exhaust temperature at 30° C.

3) Mount two Graco guns at equal distance on the boom; install hydraulic nozzles (Spraying Systems 11001-SS tips) onto the Graco guns; connect a Graco pump (piston pump) to the suspension supply line; adjust the suspension spray pressure at a pressure of 80-100 psi in order to produce a fan shape spray that covers the entire tablet bed; with the pan rotating at 10 rpm, spray incremental shots of MPA filler suspension until an average tablet weight gain of 106 mg is achieved; each shot is followed by a tumbling jog cycle of 180-300 seconds (no air through the coating pan) followed by a drying phase of 60-180 seconds.

### Color Suspensions Preparation

1) Add Water, purified in an appropriately sized jacketed container; while mixing with a high shear mixer, heat the water to 65° C.±5° C. and add the Sucrose; reheat to 65° C.; continue stirring until all the sucrose is dissolved.

2) Add the Povidone and Titanium Dioxide; mix using a high shear mixer to insure homogeneous suspension.

3) Add Cab-O-Sil and mix using a high shear mixer to make a homogeneous suspension.

4) Cool the suspension to 35-39° C.

5) Mix continuously using the low shear mixer, while maintaining the tank temperature at 35° C. to 39° C. during the entire application process.

### Color Suspension Application

1) To a 24″ perforated coating pan installed in the Comp-U-Lab coater with the special designed baffles, load approximately 33,333 Premarin/MPA filled tablets.

2) Set inlet temperature at 40° C. and inlet airflow at 75 cfm; preheat the tablets to about 30° C., dew point 11° C. and exhaust temperature at 35° C.

3) With the pan rotating at 18 rpm, apply incremental shots of color suspension until an average tablet weight gain of 25 mg is achieved; each shot is followed by a tumbling jog cycle of 180-300 seconds (no air through the coating pan) followed by a drying phase of 60-180 seconds.

### Preparation and Application of Polish

1) Prepare the polish suspension by suspending the Carnauba Wax, NF, #120 in the Mineral Spirits, Odorless with vigorous stirring.

2) Apply the polish suspension to the rolling tablets; continue rolling until a satisfactory gloss is obtained.

### Physical Appearance Evaluation and Tablet Cracking Percentage

The physical appearance of tablets was examined by observing the surface of the tablets visually, or with magnifying glasses, for surface anomalies during the coating run. In most cases, sugar coating is intended to improve the appearance of the tablets. The quality of any subsequent color and polishing processes is highly dependent upon the uniformity of the substrate filler coat. Thus, it is important to ensure that filled tablets are not cracked or chipped.

The physical appearance and percentage of cracked tablets of the batches manufactured with the different baffles was evaluated. The results are presented in Table 9. Tablets coated using the coating of the present invention and the Baffle of FIGS. 1 to 5 produced tablets of elegant appearance and low percentage of cracked tablets.

For the tablet cracking studies, coated tablets were allowed to slide down a tube into a stainless steel beaker. This process was repeated four times. Subsequently, the sugar coatings were examined for the percent of crack. The results are reported in Table 9 above.

TABLE 9
Physical Appearance, Cracking Rate, and Weight Variation of
Tablets Coated at 106 mg Targeted Weight Gain

| Physical | Cracking Rate | Wt Variation |
|---|---|---|
| Appearance | (%) | (%) |
| Excellent | 3 | 2.26 |

The disclosures of each patent, patent application and publication, including books, cited or described in this document are incorporated herein by reference in their entirety. Various modifications of the invention, in addition to those described herein, will be apparent to those skilled in the art from the foregoing description. Such modifications also are intended to fall within the scope of the appended claims.

What is claimed is:

1. A solid dosage form comprising a core material and at least one coating disposed thereon, wherein the coating comprises:
from about 30 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, at least one plasticizer in an amount of up to about 5 weight %;
optionally, a glidant, in an amount of up to about 3 weight %; and
optionally, a therapeutic agent in an amount of up to about 10 weight %.

2. The solid dosage form of claim 1, wherein the coating comprises:
from about 70 weight % to about 95 weight % of at least one sugar;
from about 0.3 weight % to about 0.8 weight % of at least one diluent/binder;
from about 0.28 weight % to about 0.4 weight % of at least one surfactant;
from about 4 weight % to about 6 weight % of at least one binder;
optionally, from about 0.5 weight % to about 1.5 weight % of at least one plasticizer;
optionally, a glidant, in an amount of up to about 1 weight %; and
optionally, a therapeutic agent in an amount of up to about 5 weight %.

3. The solid dosage form of claim 1, wherein the coating comprises:
from about 87 weight % to about 94 weight % of at least one sugar;
from about 0.4 weight % to about 0.6 weight % of at least one diluent/binder;
from about 0.28 weight % to about 0.32 weight % of at least one surfactant;
from about 4 weight % to about 6 weight % of at least one binder;
from about 0.5 weight % to about 1.5 weight % of at least one plasticizer;
optionally, a glidant, in an amount of up to about 1 weight %; and
optionally, a therapeutic agent in an amount of up to about 5 weight %.

4. The solid dosage form of claim 1, wherein in the coating, the ratio of the weight percent of binder to the weight percent of diluent/binder is from about 8:1 to about 12:1.

5. The solid dosage form of claim 1, wherein in the coating, the ratio of the weight percent of binder to the weight percent of surfactant is from about 12:1 to about 20:1.

6. The solid dosage form of claim 1, wherein in the coating, the ratio of the weight percent of binder to the weight percent of surfactant is from about 15:1 to about 18:1.

7. The solid dosage form of claim 1, wherein in the coating, the ratio of the weight percent of diluent/binder to the weight percent of surfactant is from about 1.2:1 to about 2:1.

8. The solid dosage form of claim 1, wherein in the coating, the ratio of the weight percent of binder; to the weight percent of surfactant; to the weight percent of diluent/binder; is about 10:0.6:1.

9. The solid dosage form according to claim 1, wherein in the coating:
the sugar comprises sucrose;
the diluent/binder comprises microcrystalline cellulose;
the surfactant comprises sodium lauryl sulfate; and
the binder comprises a polyvinylpyrrolidone.

10. The solid dosage form according to claim 2, wherein in the coating:
the sugar comprises sucrose;
the diluent/binder comprises microcrystalline cellulose;
the surfactant comprises sodium lauryl sulfate; and
the binder comprises a polyvinylpyrrolidone.

11. The solid dosage form according to claim 3, wherein in the coating:
the sugar comprises sucrose;
the diluent/binder comprises microcrystalline cellulose;
the surfactant comprises sodium lauryl sulfate; and
the binder comprises a polyvinylpyrrolidone.

12. The solid dosage form according to claim 8, wherein in the coating:
the sugar comprises sucrose;
the diluent/binder comprises microcrystalline cellulose;
the surfactant comprises sodium lauryl sulfate; and
the binder comprises a polyvinylpyrrolidone.

13. The solid dosage form according to claim 1, wherein in the coating:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

14. The solid dosage form according to claim 2, wherein in the coating:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

15. The solid dosage form according to claim 3, wherein in the coating:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

16. The solid dosage form according to claim 8, wherein in the coating:
the sugar comprises sucrose;
the doluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

17. The solid dosage form according to claim 1, wherein the therapeutic agent comprises a progestin, and wherein the core material comprises conjugated estrogens.

18. A solid dosage form comprising a core material and at least one coating disposed thereon; wherein the coating comprises:
from about 87 weight % to about 94 weight % of at least one sugar;
from about 0.4 weight % to about 0.6 weight % of microcrystalline cellulose;
from about 0.28 weight % to about 0.32 weight % of sodium lauryl sulfate;
from about 4 weight % to about 6 weight % of a polyvinylpyrrolidone;
from about 0.5 weight % to about 1.5 weight % of at least one plasticizer;
optionally, a glidant, in an amount of up to about 1 weight %; and
medroxyprogesterone acetate in an amount of up to about 5 weight %; and wherein the core material comprises conjugated estrogens.

19. An aqueous composition comprising water and a solids component, wherein the solids component comprises:
at least one sugar, in an amount of from about 30 weight % to about 95 weight % of the solids component;
at least one diluent/binder, in an amount of from about 0.3 weight % to about 0.8 weight % of the solids component;
at least one surfactant, in an amount of from about 0.28 weight % to about 0.4 weight % of the solids component;
at least one binder, in an amount of from about 4 weight % to about 6 weight % of the solids component;
optionally, at least one plasticizer, in an amount of up to about 5 weight % the solids component;
optionally, at least one glidant, in an amount of up to about 3 weight % the solids component; and
optionally, a therapeutic agent, in an amount of up to about 10 weight % the solids component;
wherein the water comprises from about 30 % to about 50% by weight of the aqueous composition.

20. The aqueous composition of claim 19, wherein the solids component comprises:
from about 70 weight % to about 95 weight % of the sugar;
from about 0.3 weight % to about 0.8 weight % of the diluent/binder;
from about 0.28 weight % to about 0.4 weight % of the surfactant;
from about 4 weight % to about 6 weight % of the binder;
optionally, from about 0.5 weight % to about 1.5 weight % of the plasticizer;
optionally, up to about 1 weight % of the glidant; and
optionally, up to about 5 weight % of the therapeutic agent.

21. The aqueous composition of claim 19, wherein the solids component comprises:
from about 87 weight % to about 94 weight % of the sugar;
from about 0.4 weight % to about 0.6 weight % of the diluent/binder;
from about 0.28 weight % to about 0.32 weight % of the surfactant;
from about 4 weight % to about 6 weight % of the binder;
optionally, from about 0.5 weight % to about 1.5 weight % of the plasticizer;
optionally, up to about 1 weight % of the glidant; and
optionally, up to about 5 weight % of the therapeutic agent.

22. The aqueous composition of claim 19, wherein, in solids component:
the sugar comprises sucrose;
the diluent/binder comprises microcrystalline cellulose;
the surfactant comprises sodium lauryl sulfate;
the binder comprises a polyvinylpyrrolidone;
the plasticizer, when present, comprises a polyethylene glycol;
the glidant, when present, comprises silica; and
the therapeutic agent, when present, comprises medroxyprogesterone acetate.

23. The aqueous composition of claim 19, wherein the plasticizer, the glidant, and the therapeutic agent are each present in the solids component.

24. A method for preparing a solid dosage form, comprising coating a core material with an aqueous composition of claim 19.

25. A product of the process of claim 24.
